(19) United States
(12) Patent Application Publication
(10) Pub. No.: US 2025/0090080 A1
(43) Pub. Date: Mar. 20, 2025

(54) SYSTEM AND METHOD FOR EVALUATING A PATIENT'S BALANCE, COORDINATION AND STABILITY TO ASSIST WITH FALL PREVENTION THERAPIES

(71) Applicant: Maurice Pisciottano, Casnonsburg, PA (US)

(72) Inventor: Maurice Pisciottano, Casnonsburg, PA (US)

(21) Appl. No.: 18/749,407

(22) Filed: Jun. 20, 2024

Related U.S. Application Data
(60) Provisional application No. 63/521,829, filed on Jun. 19, 2023.

(51) Int. Cl. A61B 5/00; A63B 24/00

(52) U.S. Cl. CPC A61B 5/4023; A61B 5/742; A63B 24/0062

(57) ABSTRACT

A system for evaluating a patient's balance, coordination and stability comprises an evaluation station with a variable stability platform situated between a pair of safety handrails; and a graphical user interface designed to receive information from the platform when a patient performs, or attempts to perform, a series of minimal balance evaluations/tests when prompted by the system. A method of using this system is also disclosed.

### CROSS-REFERENCE TO RELATED APPLICATIONS

This application is a perfection of U.S. Provisional Application Ser. No. 63/521,829, filed on Jun. 19, 2023, the disclosure of which is fully incorporated by reference herein.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable.

### STATEMENT REGARDING PRIOR DISCLOSURES BY THE INVENTOR OR A JOINT INVENTOR

Not Applicable.

### BACKGROUND OF THE INVENTION

### (1) Field of the Invention

This invention relates to the field of patient testing and evaluation. Particularly, it relates to a system and method for evaluating a patient's balance, coordination and stability. The system and method of this invention, made and marketed under the PROBalance 360™ trademarks and service marks, enables an operator to perform vestibular testing and vestibular therapies.

From Harvard Medical School, there are five major systems of the human body that contribute to balance: the visual system, the hearing system, the peripheral nervous system, the central nervous system and the musculoskeletal system. For the individual patient, balance issues manifest in many ways. That's why the patient must first ask him or herself, critically, whether he/she has experienced any one or more of the following symptoms or ailments: dizziness or lightheadedness, frequent headaches or migraines, frequent falls, sudden loss of hearing, sensitivity to light or sound, stroke, vertigo, concussion and/or head or neck trauma. The patient is asked to rate his/her own balance and stability on a scale from one to ten. Next, the patient checks off on how often he/she experiences balance or stability problems: constantly, daily, weekly, monthly. Then, the patient should advise the operator of this system what steps have been taken to alleviate these systems and/or restore balance. They can range from rest alone, ice, heat, massage, physical therapy, and/or drug related, i.e., pain killers, muscle relaxants, other OTC medicines, etc.

This invention allows an operator to first test an individual's baseline balance by going through a series of specific measures on a variable stability platform while being observed with a graphic user interface, said measures being tailored to test for any directional balance deficits. After initial evaluation, the patient may have a series of therapeutic exercises performed on key body areas, e.g., hip, knee, shoulder, etc. before being retested to observe for balance improvements. An underlying goal of this invention is to not only assist with particular patient balance observations, but to use targeted therapies for improving a given patient's stabilities while further reducing his/her risks of falling-i.e., fall prevention.

### (2) Description of Related Art Including Information Disclosed Under 37 CFR 1.97 and 1.98

Known art relevant to this invention which it nevertheless distinguishes over, includes but is not limited to the following disclosures arranged in chronological order: U.S Published Application Nos. 20060122579, 20160045393 and 20220287625 together with U.S. Pat. Nos. 8,641,648, 9,149,222, 10,376,739, 11,213,244 and 11,389,083.

### BRIEF SUMMARY OF THE INVENTION

The system of this invention comprises an evaluation station with a variable stability platform situated between a pair of safety handrails. The platform faces a display screen connected to a graphical user interface designed to receive information from the platform when a patient performs, or attempts to perform, a series of balance evaluations/tests when prompted by the system and its individual operator. The minimal evaluations to be performed should include a plurality of balance evaluative exercises when the patient: (a) stands at rest on the platform, eyes open; (b) stands, eyes closed; (c) left leg raised, eyes open; (d) left leg raised, eyes closed: (e) right leg raised, eyes open; and (f) right leg raised, eyes closed.

For each evaluation, the system will perform a determination on a patient's stability index. It will generate on a display for the patient to see, a 2-dimensional depiction graphing the center point onscreen and how far the patient wavered from a centermost point.

The evaluation will also provide the operator with information on how muscular exercising/stimulating devices like the PROSoft GT may be placed on various external body parts for affecting a change to that patient's balance, posture, coordination, proprioception, or altogether overall stability.

The system of this invention may be further used for the patient to graphically exercise his/her own rotational movements, on a timed test basis, for helping to better score improved stability on one or more follow up evaluations. These graphic exercises have been couched in terms of “games” more than rigorous exercises per se.

### BRIEF DESCRIPTION OF THE SEVERAL VIEWS OF THE DRAWINGS

Further features, improvements and developments for the present invention will become clearer when reviewing the accompanying description below, made with reference to the photographs in which:

FIG. 1 is a right rear perspective view of an individual (patient P) standing on the platform component PC of this system S, between safety handrails HR1 and 2 with the graphic user interface screen GUI on a homepage HP for the operator (not shown) to next choose between evaluation E or therapy T modes. Note that a PROSoft GT exercising device PSD is also shown prior to use on a patient;

FIG. 2 is a rear view of an individual (patient P) standing with both legs on the system's platform component PC with a representative evaluation graphic (in this case, a circular quadrant display CQD, a representative type of stability indicator) on the GUI display;

FIG. 3A is a front view of the individual patient P on the system S (as seen from behind the graphic user interface GUI on its stand), said individual having her eyes open and left leg raised in this representative view for “initial” stability evaluation purposes;

FIG. 3B is a rear view of the same individual patient P from FIG. 3A, her left leg raised, for evaluation by the system S;

FIG. 4A is a front view of the same individual patient P on the system S (as seen from behind the graphic user interface GUI), said individual patient P having her eyes open and right leg “raised”, (or bent to about a ninety-degree angle) for evaluation purposes;

FIG. 4B is a rear view of the same individual patient P, her right leg raised;

FIG. 5 is a close up showing the same individual patient P with her eyes closed in order to perform one or more of the preceding stability evaluations on the platform component PC of the system S;

FIG. 6 is a front view of the graphic user interface GUI, with its 2-dimensional circular quadrant display CQD before being used for a patient evaluation. Note, the “bullseye” location B of the evaluating system's cursor SC on the display screen; and

FIG. 7 is a front view of the graphic user interface when the system S is in mid-use on an individual. (not shown). Note how the system's cursor SC is positioned at about the 9 o'clock point on the display screen.

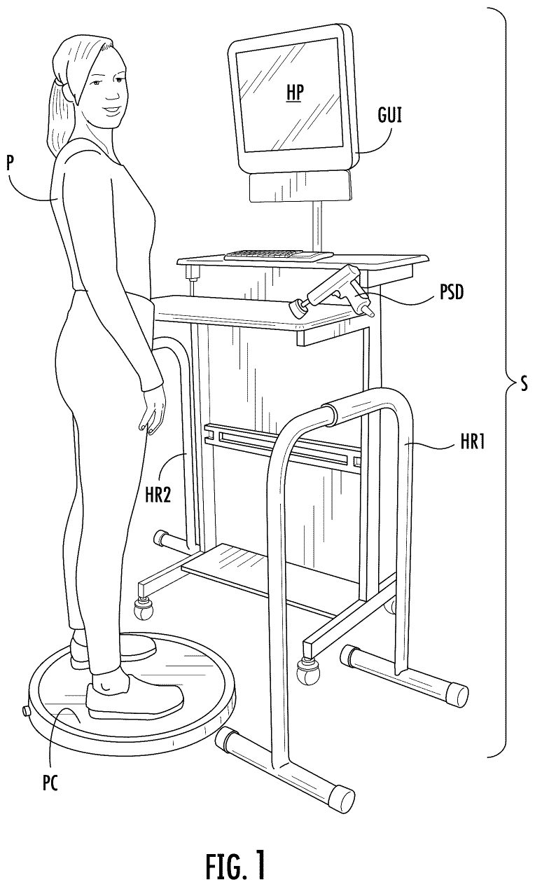

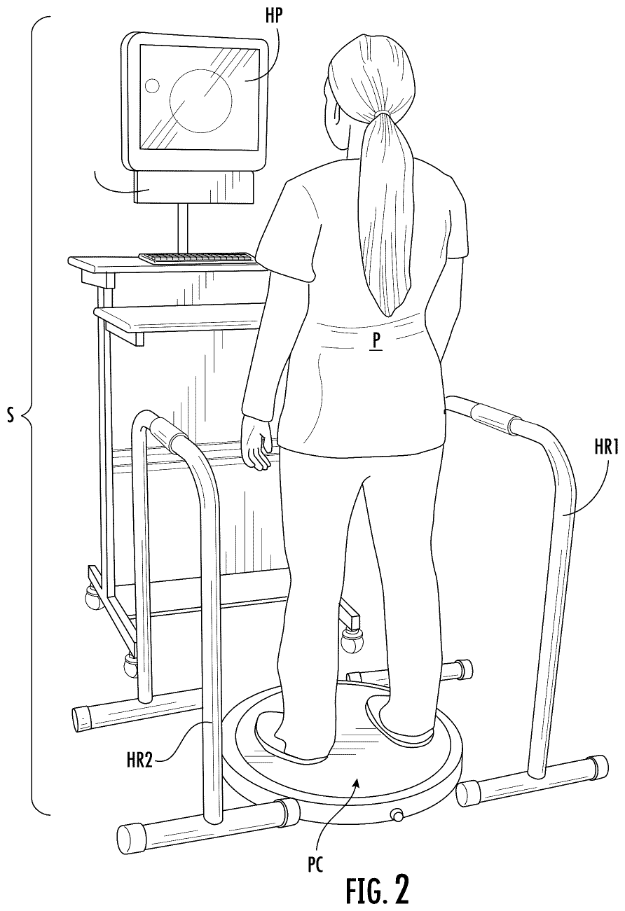

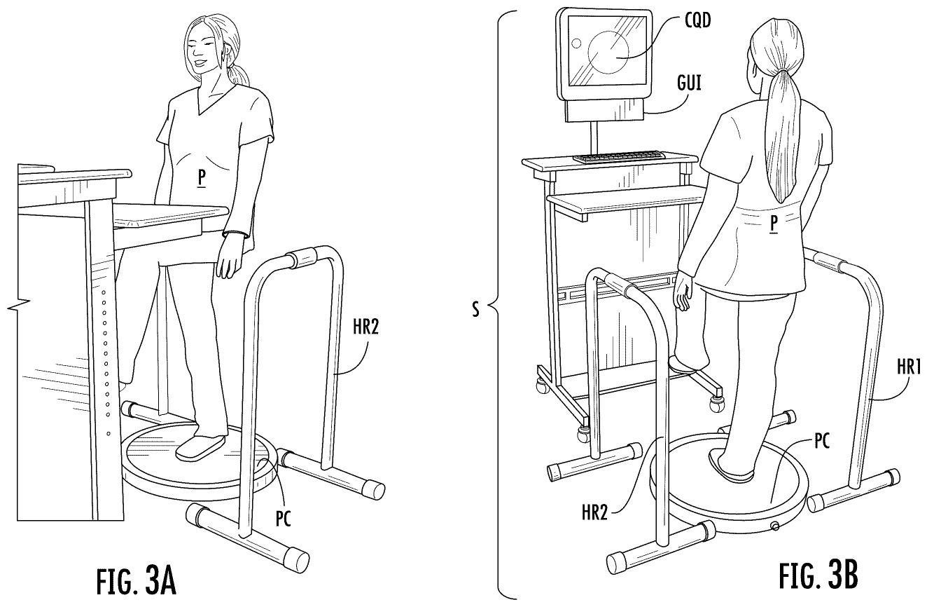

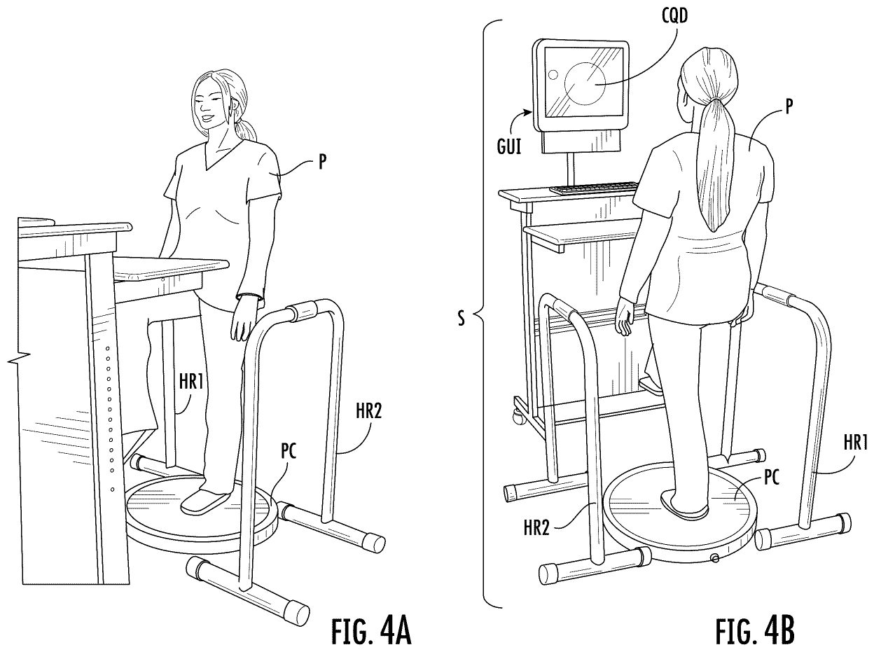

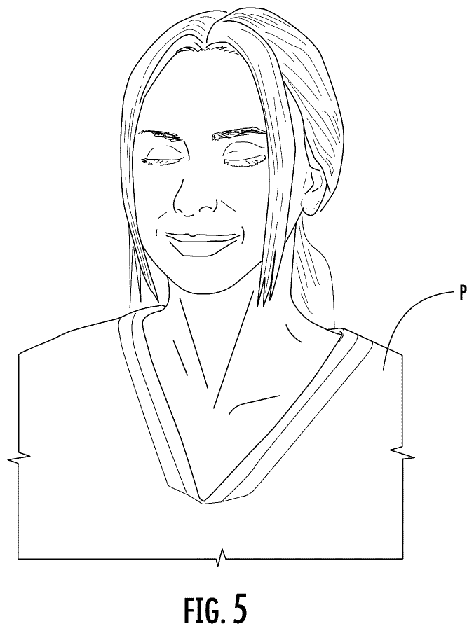

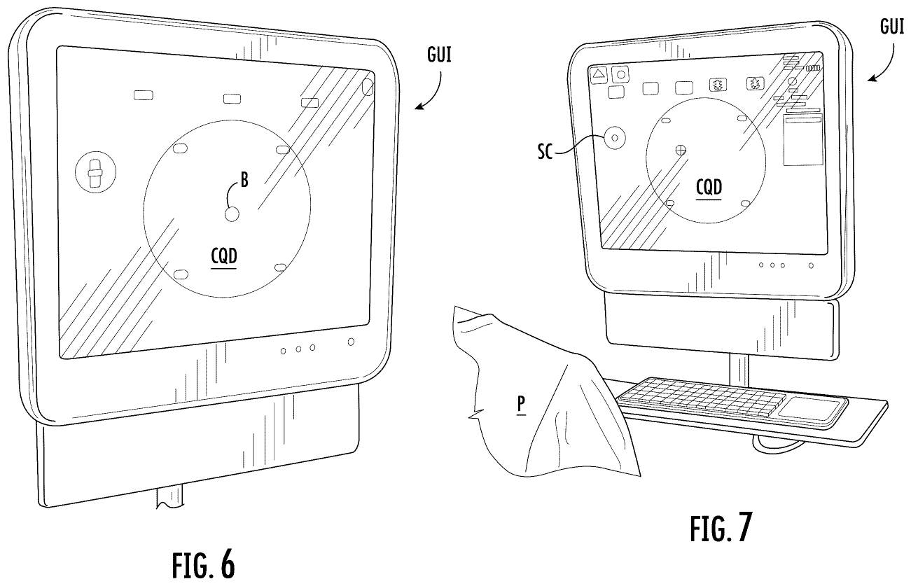

### DETAILED DESCRIPTION OF THE INVENTION

This invention addresses a new Balance and Fall Prevention Therapy with the ProBalance360™ system. It also addresses affiliated methods for using the system to perform Mechanical Motion Therapy (or MMT) sessions on an individual/patient. The system and method allow an operator to evaluate a patient's key balance metrics, and use that information to develop a customized therapeutic plan designed to strengthen the musculoskeletal system and rehabilitate the neural pathways to improve his/her overall balance, coordination, and stability.

The system/method includes the following “steps”:

### Step 1: Evaluation

An initial (or baseline) evaluation will be performed to determine a starting Stability Function Index (SFI) for a given individual. That information will be used to create a customized treatment plan designed to: (i) strengthen key musculoskeletal systems; and (ii) correct proprioceptive deficits throughout the body.

### Step 2: Treatment

Mechanical Motion Therapy (MMT) and/or Vestibular Neuropathway Therapy (VNT) use specialized protocols to stimulate the soft tissues and neural pathways, leading to restored and enhanced balance and coordination by correcting problems that result from musculoskeletal and proprioceptive deficits.

### Step 3: Freedom

Whether the individual seeks an edge on the field, or simply wants to improve his/her quality of life, Mechanical Motion Therapy (MMT) and/or Vestibular Neuropathway Therapy (VNT) help thousands of patients around the world improve performance, restore peace of mind, and safely lead the lifestyle they want to live. MMT/VNT can help reduce pain, increase functional mobility and restore balance and coordination by targeting special receptors in the soft tissues of the body. Patient goals include overcoming one's neck pain, back pain, hip pain, shoulder pain, knee pain, arm pain, foot pain and/or headaches. With MMT/VNT, the individual should be able to:

### Feel Better and Function Higher!

The present system and method sense neuromuscular, biomechanical, and musculoskeletal activity of an individual and, in some instances, provide feedback related to fall prevention based on sensed information.

The system and method described herein are configured for collecting valid and reliable data outside of a laboratory environment. As such, these devices, systems, and methods are capable of providing more sophisticated analysis and feedback concerning movement of a subject than is provided using conventional fitness monitor devices. In one example, the collected data is used for providing predictive feedback related to fall risk and recommended training regimens based on collected movement information.

Exemplary musculoskeletal assessments include assessments of range of motion and flexibility of certain muscle groups and joints including, for example, the ankle, knee, hip, back, and/or shoulders, Assessments can also include an assessment of strength and endurance of the ankle, knee, hip, back, and/or shoulder musculature. Biomechanical assessments include, for example, an assessment of joint kinematic and kinetic data for the ankle, knee, hip, back, and/or shoulders, including maximum values or ranges of values for angles of movement, movement velocities, and accelerations, or for impact forces and ground reaction forces experienced during sport, military training, occupational, and common daily activities. Neuromuscular assessments include, for example, assessments of one or more of the following: lower extremity, upper extremity, and/or back motor control; balance and/or postural stability including dynamic postural stability; and joint coordination.

An itemized subject report SR is prepared from the collected and analyzed data. For example, assessment results, derived values, predictive injury information, and training recommendations generated based on data that can be collected into a formal hardcopy report. The report can be a basic report which merely includes certain derived measures or a comprehensive report showing derived measures, associated risk, and training recommendations. For example, a comprehensive report can include a combination of raw data, graphs, and analyzed results, such as predictive fall risk information.

Once the subject is in position, the user selects the begin evaluation button. The user or subject is next provided with a list of possible balance measurements that are taken by the wearable sensor device. In this example, the user or subject selects from one or more of the following: double leg static stance with eyes open or with eyes closed; single leg static stance with eyes open or eyes closed; and dynamic postural stability.

Having described the best modes currently known for practicing this system and method, it is to be understood that the scope of this invention may be further described by the attached claims.

### SEQUENCE LISTING

Not applicable.

1. A system for evaluating a patient's balance, coordination and stability comprising:
an evaluation station with a variable stability platform situated between a pair of safety handrails; and a graphical user interface designed to receive information from the platform when a patient performs, or attempts to perform, a series of minimal balance evaluations/tests when prompted by the system.

2. The evaluation system of claim 1 wherein the series of minimal balance evaluations to be performed include the patient: (a) standing at rest on the variable stability platform, with eyes open; (b) standing, with eyes closed; (c) left leg raised, with eyes open; (d) left leg raised, with eyes closed: (e) right leg raised, with eyes open; and (f) right leg raised, with eyes closed.

3. The evaluation system of claim 1 which generates a report quantifying a stability index for the patient.

4. The evaluation system of claim 1 wherein the graphical user interface generates a 2-dimensional depiction graphing the center point onscreen and how far the patient wavered from a centermost point.

5. The evaluation system of claim 1 that will provide an operator on how muscular exercising/stimulating devices may be placed on various external body parts to affect a change to that patient's balance, posture, coordination, and/or proprioception, i.e., overall stability.

6. A system for initially evaluating a patient's balance, coordination and stability,
then monitoring the patient's exercises to improve at least one of the patient's
balance, coordination and stability, said system comprising:
a patient evaluation station that includes: a variable stability platform situated between a pair of safety handrails; and a graphical user interface designed to receive information from the platform when a patient performs, or attempts to perform, a series of minimal balance evaluations/tests when prompted by the system, said graphical user interface including means for mapping the patient's exercises to improve at least one of the patient's balance, coordination and stability.

7. The system of claim 6 wherein the series of minimal balance evaluations to be performed include the patient: (a) standing at rest on the variable stability platform, with eyes open; (b) standing, with eyes closed; (c) left leg raised, with eyes open; (d) left leg raised, with eyes closed: (e) right leg raised, with eyes open; and (f) right leg raised, with eyes closed.

8. The evaluation system of claim 7 which generates a report quantifying a stability index for the patient.

9. The evaluation system of claim 7 wherein the graphical user interface generates a 2-dimensional depiction graphing the center point onscreen and how far the patient wavered from a centermost point.

10. The evaluation system of claim 7 that will provide an operator on how muscular exercising/stimulating devices may be placed on various external body parts to affect a change to that patient's balance, posture, coordination, and/or proprioception, i.e., overall stability.

11. A method for evaluating a patient's balance, coordination and stability, said method comprising:
(a) providing an evaluation station with a variable stability platform situated between a pair of safety handrails; and a graphical user interface designed to receive information from the platform when a patient performs, or attempts to perform, a series of balance evaluations/tests when prompted by the system;
(b) situating the patient on the platform to perform the series of balance evaluations/tests; and
(c) generating a report quantifying a stability index for the patient.

12. The method of claim 11, which further comprises:
(d) performing a plurality of muscular exercising/stimulating devices on various external body parts to affect a change to that patient's balance, posture, coordination, and/or proprioception, i.e., overall stability; and
(e) re-evaluating the patient at the evaluation station.

13. The method of claim 12 wherein the series of minimal balance evaluations to be performed include the patient: (a) standing at rest on the variable stability platform, with eyes open; (b) standing, with eyes closed; (c) left leg raised, with eyes open; (d) left leg raised, with eyes closed: (e) right leg raised, with eyes open; and (f) right leg raised, with eyes closed.

14. The method of claim 12 which generates a report quantifying a stability index for the patient.

15. The method of claim 12 wherein the graphical user interface generates a 2-dimensional depiction graphing the center point onscreen and how far the patient wavered from a centermost point.

16. The method of claim 12 that will provide an operator on how muscular exercising/stimulating devices may be placed on various external body parts to affect a change to that patient's balance, posture, coordination, and/or proprioception, i.e., overall stability.
